(19) United States
(12) Patent Application Publication
(10) Pub. No.: US 2011/0046214 A1
(43) Pub. Date: Feb. 24, 2011

(54) COMPOSITION INHIBITING SEX HORMONE-BINDING GLOBULIN

(76) Inventors: Shaw WATANABE, Setagaya-ku (JP); Hideo Araki, Izumisano-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/845,008

(22) Filed: Jul. 28, 2010

Related U.S. Application Data
(62) Division of application No. 11/664,387, filed on May 1, 2007, now abandoned.

(30) Foreign Application Priority Data
Sep. 30, 2004 (JP) 2004-286784

(51) Int. Cl. A61K 31/352; A61P 3/00

(52) U.S. Cl. 514/456

(57) ABSTRACT

A composition for inhibiting sex hormone-binding globulin, comprising isoflavones as active ingredients.

### TECHNICAL FIELD

The present invention relates to a composition for inhibiting sex hormone-binding globulin, and relates to a composition for alleviating or improving various symptoms due to the sex hormone-binding globulin inhibiting effect.

### BACKGROUND ART

With rapid progress toward aging society, the medical cost and the like are increasing, and such problems as collapse of health insurance system and oppression of finances are occurring. Therefore health maintenance by improvement of lifestyle habit such as eating habit, without relying on drugs, is sought not only in the aged but also in every generation.

In East Asian countries including Japan, soybeans have been eaten since thousands of years ago, and have been protein sources useful for maintaining health.

In recent years, the relationship between isoflavones contained in soybeans, and climacteric symptoms, prevention of osteoporosis, breast cancer, or prostate cancer has been reported. Thus, isoflavones have attracted attention as food components beneficial to middle-aged and older people (see New Food Ind., Vol.40, No.8, 9-14,1998).

Currently, it is believed that isoflavones competitively inhibit endogenous estrogen, and stimulate production of sex hormone-binding globulin (hereinafter, referred to as “SHBG”) to increase the blood level of SHBG. In addition, a cell test showed that genistein stimulates production of SHBG (see Steroids, vol.58, July, 301-304, 1993). That is, it has been believed that isoflavones promote production of SHBG, and then the increased SHBG binds to endogenous estrogen to control it, thereby improving the aforementioned symptoms. However, said action of isoflavones has never been demonstrated actually in clinical tests, and the aforementioned action mechanism has not been confirmed.

### DISCLOSURE OF THE INVENTION

### Problems to be Solved by the Invention

An object of the present invention is to find out a novel use of isoflavones which are derived from natural products and are highly safe even when added to foods, and to use them to provide a composition for improving various symptoms.

### Means for Solving the Problems

In view of the above problems, the inventors of the present invention intensively studied influence of isoflavones, which are contained in a large amount in leguminous plants such as soybean and clover, ingested by a human on the blood hormone levels etc. and, as a result, found that isoflavones inhibit SHBG, the blood level of which had been thought to be increased by isoflavone ingestion, and said inhibitory activity promotes secretion of estradiol, which is one of endogenous hormones. The inventors of the present invention further studied and, as a result, found that active ingredients exerting particularly remarkable effects among isoflavones are those of the daidzein group, which are contained in a large amount in the hypocotyls of soybeans.

That is, the present invention discloses:

1. A composition for inhibiting sex hormone-binding globulin, comprising isoflavones as active ingredients;
2. The composition according to the above 1, wherein the isoflavones contain one or more kinds of isoflavones selected from the daidzein group;
3. The composition according to the above 1, wherein a supply source of the active ingredient is an isoflavone-containing material originating from a plant;
4. The composition according to the above 3, wherein the weight rate of daidzein group/genistein group present in the isoflavone-containing material originating from a plant is 2 or more;
5. The composition according to the above 4, wherein the isoflavone-containing material originating from a plant is derived from a soybean hypocotyl;
6. The composition according to the above 5, wherein the isoflavone-containing material originating from a plant is an extract derived from a soybean hypocotyl or a purified product thereof;
7. The composition according to the above 1, which is a food product or an agent;
8. The composition according to the above 1, which is a food product or a medicinal product with an indication of health product based on the sex hormone-binding globulin inhibitory activity; and
9. Use of isoflavones for the preparation of a composition for inhibiting sex hormone-binding globulin.

### EFFECT OF THE INVENTION

Use of the composition of the present invention enables inhibition of blood SHBG, and thereby, an increase in the blood SHBG level with aging can be suppressed to promote secretion of an endogenous hormone such as estradiol into blood. Further, on the basis of such an effect, the present invention can provide a composition expected to have effects on various symptoms, for example, alleviation of climacteric symptoms, prevention of osteoporosis, improvement of fat metabolism, improvement in brain functions and the like, and therefore may greatly contribute to health maintenance in modern people.

### BEST MODE FOR CARRYING OUT THE INVENTION

The composition for inhibiting sex hormone-binding globulin of the present invention is characterized by isoflavones as the active ingredients.

In the present invention, isoflavones are contained in a large amount in plants such as soybean, red clover, purple clover and the like, and are classified based on their structures. Their examples include the daidzein group which has daidzein as the aglycon skeleton (daidzein which is aglycon form, daidzin in which glucose is β-bound to the aglycon skeleton, malonyldaidzin, acetyldaidzin and succinyldaidzin in which a functional group is bound to the glucose part of daidzin, etc.), the genistein group which has genistein as the aglycon skeleton (genistein, genistin, malonylgenistin, acetylgenistin, succinylgenistin etc.), the glycitein group which has glycitein as the aglycon skeleton (glycitein, glycitin, malonylglycitin, acetylglycitin, succinylglycitin etc.), biokanin A, formononetin and the like. In addition, equol, which is a metabolite of the daidzin group, is also included. One or more kinds of them can be selected.

As the active ingredient, a purified pure product may be used, or an isoflavone-containing material originating from a plant which is prepared from a plant raw material such as soybean, black soybean, purple clover or clover as its supply source may be used. For exerting the effect of the active ingredient more remarkably, the isoflavone-containing material originating from a plant is preferably an isoflavone-containing material originating from a plant in which the daidzein group/genistein group are present at a weight rate of 2 or more. Specific examples of the isoflavone-containing material originating from a plant include isoflavone-containing materials derived from soybean hypocotyls. When the isoflavone-containing material is used, a method of preparing the material from the aforementioned leguminous plants is not particularly limited, and for example, a method comprising grinding of the aforementioned leguminous plants, a method comprising extraction of the aforementioned leguminous plants with water, an organic solvent such as ethanol or acetone, or a mixture thereof, a method comprising purification of the extract using an adsorbent resin, an ion exchange resin or the like, or a method comprising purification by liquid-liquid distribution with an organic solvent can be used. For exerting the effect of the active ingredient more remarkably, it is more preferable to use an extract from soybean hypocotyls, or a purified product thereof.

The active ingredient of the present invention may be subjected to processing treatment for imparting various properties thereto. For example, the active ingredient may be included in cyclodextrin in order to enhance solubility, or soluble saccharides such as glucose may be α-bound to the active ingredient. Alternatively, all of the active ingredients may be converted into the aglycon forms by the action of β-glucosidase, or such enzyme reaction may be attained by fermentation with koji, yeast, lactic acid bacteria, Bacillus natto or the like.

The content of the active ingredient in the composition of the present invention varies depending on the form and amount of the composition, and can be appropriately determined. Usually, a person skilled in the art may determine the content of the active ingredient in the composition so that the daily ingested amount of the active ingredient can be ingested, in consideration of the daily ingested amount of the composition. For example, in the case where the daily ingested amount of the active ingredient is 10 mg and the daily ingested amount of the composition is 100 g, the content of the active ingredient in the composition may be 0.01% by weight.

Upon preparation of the composition of the present invention, depending on the purpose, raw materials containing useful ingredients derived form animals or plants such as protein, lipid, sugar, dietary fiber, oligosaccharide, amino acid, peptide, mineral, vitamin, catechin, ginkgo leaf, anthocyanin, GABA, L-carnitine, α-lipoic acid, or saponins such as soybean saponin; useful ingredients derived form microorganisms such as yeast extract, polyglutamic acid, Bacillus natto powder, or lactic acid bacterium powder; or coenzymes such as Coenzyme Q10, enzymes, or the like in large amounts can be used in combination with the active ingredient of the present invention.

The composition of the present invention refers to a food product or an agent. In the case of the agent, the composition can be formulated into preparations in various dosage forms. That is, in the case of oral administration, the composition can be administered in the form of a solid preparation such as tablet, hard capsule, soft capsule, granule or pill, or a liquid preparation such as solution, emulsion or suspension. In the case of parenteral administration, the composition is administered in the form of an injection solution, a suppository or the like. Upon preparation of these agents, pharmaceutically acceptable additives such as excipient, stabilizer, antiseptic, wetting agent, emulsifier, lubricant, sweetener, colorant, flavor, tonicity adjusting agent, buffer, antioxidant, pH adjusting agent and the like can be used in combination with the aforementioned ingredients to formulate the composition into the aforementioned dosage form.

When the composition of the present invention is a food product, the composition can be incorporated into various food products such as soft drinks, dairy products, soybean milk, fermented soybean milk, soybean protein drinks, bean curd (tofu), fermented soybeans (natto), deep-fried bean curd, thick deep-fried bean curd, deep-fried bean curd containing bits of various kinds of vegetables (ganmodoki), hamburgers, meat balls, deep-fried fish or chicken, fish or chicken nuggets, various side dishes, confectionary such as baked confectionary, cereals, candies, gums and the like, tablets, breads, cooked rice and the like. Further, since the content of the active ingredient (isoflavones) in food products can be easily measured, the composition of the present invention can be also a food product for health (such as a specified health food product or the like) on the package, pamphlet or the like for which there is a description indicating that said food product has various efficacies and effects based on inhibition of sex hormone-binding globulin because said food products contain isoflavones as the active ingredients (the ingredients involved in the efficacies and effects).

The effective ingested amount of the sex hormone-binding globulin inhibitor or food product thus obtained varies depending on the use purpose, the subject to be administered and the form of use. In the case of a human, usually, the amount may be adjusted so that about 10 to 500 mg of the active ingredient per day can be ingested, and may be ingested once a day or in several divided doses per day. Many medicaments may cause safety problems by ingestion of an amount larger than the proper amount. In contrast, in the case of the composition of the present invention, the upper limit of the ingested amount matters little from a viewpoint of safety because a natural isoflavone-containing material derived from a plant can be used as the active ingredient of the present invention.

Hereinafter, examples of the present invention will be shown, but the technical scope of the present invention is not limited to them.

### EXAMPLES

### Example 1

### Preparation of Sex Hormone-Binding Globulin Inhibitory Tablet

(Preparation of isoflavone-containing material)

According to the method of JP Patent No.3191799, an isoflavone-containing material was prepared from soybean hypocotyls as follows. Soybean hypocotyls were dry-heated, immersed in water to remove unnecessary polysaccharides, and then extracted with hot water to obtain a soybean hypocotyl extract. The extract was powderized with a spray drier to obtain an isoflavone-containing material. The daidzein group/genistein group rate of the resulting isoflavone-containing material was 3.8. The total content of isoflavones of the daidzein group and the genistein group in the resulting isoflavone-containing material was 5.3% by dry weight. The total isoflavone content including the glycitein group was 8.3%.

(Preparation of test sample)

A test tablet was prepared using the above-described isoflavone-containing material. Lactose was mixed with the isoflavone-containing material in a total isoflavone aglycon amount of 7 mg per 1 g of the total weight of raw materials. The mixture was compressed to prepare a test tablet (0.285 g per tablet). The test tablet was designed to be administered in a dose of 20 tablets per day, and the daily dose contained an isoflavone aglycon amount of 40 mg and a total isoflavone amount of 68 mg.

### Comparative Example 1

### Preparation of Placebo Tablet

As a placebo tablet used as a control, a tablet in which the isoflavone-containing material of Example 1 was replaced with lactose was prepared.

(Test method)

In 45 menopausal females and 13 premenopausal females as subjects, an intervention test was performed using the tablet (IF tablet) of the present invention obtained in Example 1 (an isoflavone aglycon amount per day of 40 mg, a daidzein group-isoflavone aglycon amount of 27 mg) and the placebo tablet obtained in Comparative Example 1. The test was performed by a double blind cross-over test in which the IF tablet and the placebo tablet were ingested each for 4 weeks. Before intervention (baseline), and 4 weeks and 8 weeks after the test, determination of climacteric symptoms, a urine test and collection of blood sample were performed, and in addition, the circumstance of the tablet ingestion was recorded. Blood samples collected on the baseline and during the ingestion terms of the IF tablet and the placebo tablet were used in measuring the hormone levels. The hormones were estradiol, progesterone, corpus luteum hormone, follicle-stimulating hormone, prolactin and SHGB and measured by a fluoroimmunometric assay using DELIFIA. As an index of climacteric symptoms, a simplified menopausal index (SMI) was used.

(Test result)

As shown in Table 1, ingestion of the IF tablet suppressed an increase in SHBG only in menopausal females as compared with before intervention, while it significantly increased the serum estradiol level. In premenopausal females, an increase of estradiol was not shown, and SHBG was not increased as compared with before intervention. There was no influence on other endogenous hormones. In addition, influence of the IF tablet ingested on climacteric symptoms in menopausal females is shown in FIG. 1. As seen from FIG. 1, climacteric symptoms in menopausal females were significantly improved (p<0.05) by ingestion of the IF tablet, as compared with before intervention. The IF tablet was found to have the effect on, in particular, hot flash among climacteric symptoms. It is thought that promotion of estradiol secretion due to the SHBG inhibitory activity of isoflavones is involved in such effect.

TABLE 1
Influence of ingestion of IF tablet
on blood SHBG and estradiol levels

| | | Before | | |
|---|---|---|---|---|
| | | intervention | IF | Placebo |
| Estradiol | pg/ml | 15.0 | 40.8 a b | 10.9 |
| Progesterone | ng/ml | 0.3 | 0.9 | 0.5 |
| Corpus | U/L | 25.2 | 28.0 | 31.9 |
| luteum | | | | |
| hormone | | | | |
| Follicle- | U/L | 61.1 | 68.0 | 73.5 |
| stimulating | | | | |
| hormone | | | | |
| Prolactin | ng/ml | 4.3 | 4.5 | 4.4 |
| SHBG | nmol/L | 63.7 | 57.5 b | 83.4 |

45 menopausal females
a: There is a significant difference relative to before intervention (p < 0.05)
b: There is a significant difference relative to placebo (p < 0.05)

From the above-described results, it was found that isoflavones are active ingredients involved in inhibition of blood SHBG and promotion of estradiol secretion. Further, the results demonstrate that, among isoflavones, isoflavones of the daidzein group are active ingredients.

### BRIEF DESCRIPTION OF DRAWINGS

FIG. 1 is a graph showing the improving effects on climacteric symptoms owing to ingestion of the IF tablet.

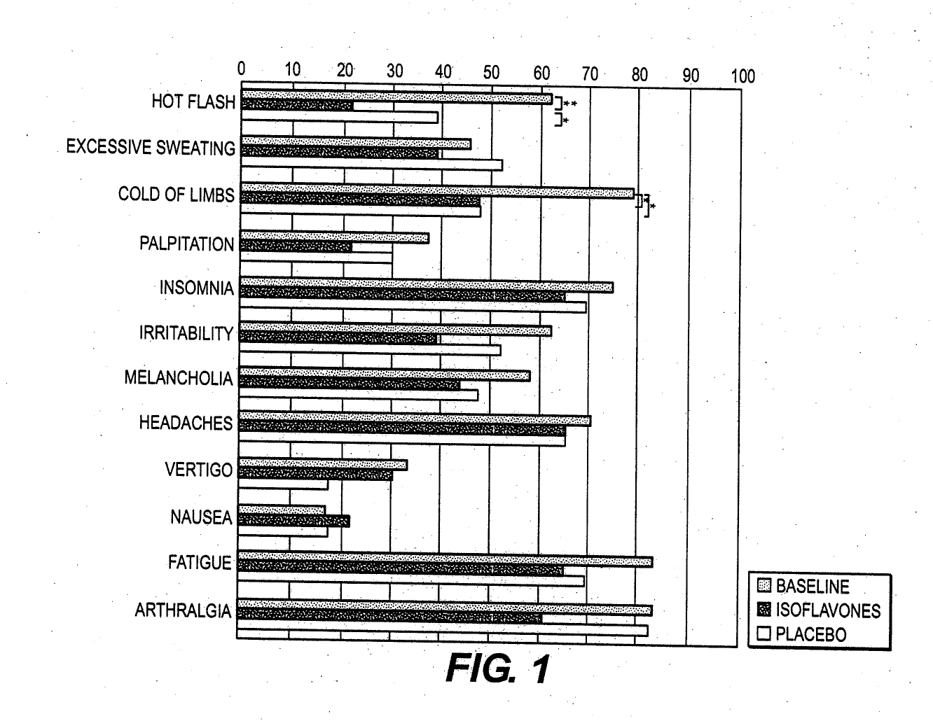

1-9. (canceled)

10. A method for inhibiting blood sex hormone-binding globulin in a female after menopause, which comprises administering to a female after menopause an isoflavone-containing material originating from a plant and having 2 or more of the weight rate of daidzein group/genistein group.

11. A method for increasing the blood level of estradiol in a female after menopause, which comprises administering to a female after menopause an isoflavone-containing material originating from a plant and having 2 or more of the weight rate of daidzein group/genistein group.
